SEQUENCE LISTING (68 sequences)

SEQ ID NO: 1 - DNA, 25 bp, artificial
  synthetic oligonucleotide primer
tttatccatcccatcacaccctcag

SEQ ID NO: 2 - DNA, 22 bp, artificial
  synthetic oligonucleotide primer
ccactgacaaccacccttaacc

SEQ ID NO: 3 - DNA, 22 bp, artificial
  synthetic oligonucleotide primer
ccgtatagcgtaaattcccaga

SEQ ID NO: 4 - DNA, 20 bp, artificial
  synthetic oligonucleotide primer
ctgttttccagggactgagg

SEQ ID NO: 5 - DNA, 23 bp, artificial
  synthetic oligonucleotide primer
caactctgtctccttcctcttcc

SEQ ID NO: 6 - DNA, 20 bp, artificial
  synthetic oligonucleotide primer
agacctcaggcggctcatag

SEQ ID NO: 7 - DNA, 24 bp, artificial
  synthetic oligonucleotide primer
ttctgaggttatctttttaccaca

SEQ ID NO: 8 - DNA, 22 bp, artificial
  synthetic oligonucleotide primer
ggaagaggaaaggaaaaacatc

SEQ ID NO: 9 - protein, 382 aa, Homo sapiens
  VARIANT (62)..(62): optional lysine to glutamic acid substitution
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 10 - DNA, 1536 bp, Homo sapiens
  variation (123)..(123): optional C to T single-nucleotide polymorphism
acagagcttcaaaaaaagagcgggacagggacaagcgtatctaagaggctgaacatgaatccacagatcagaaatccgatggagcggatgtatcgagacacattctacgacaactttgaaaacgaacccatcctctatggtcggagctacacttggctgtgctatgaagtgaaaataaagaggggccgctcaaatctcctttgggacacaggggtctttcgaggccaggtgtatttcaagcctcagtaccacgcagaaatgtgcttcctctcttggttctgtggcaaccagctgcctgcttacaagtgtttccagatcacctggtttgtatcctggaccccctgcccggactgtgtggcgaagctggccgaattcctgtctgagcaccccaatgtcaccctgaccatctctgccgcccgcctctactactactgggaaagagattaccgaagggcgctctgcaggctgagtcaggcaggagcccgcgtgacgatcatggactatgaagaatttgcatactgctgggaaaactttgtgtacaatgaaggtcagcaattcatgccttggtacaaattcgatgaaaattatgcattcctgcaccgcacgctaaaggagattctcagatacctgatggatccagacacattcactttcaactttaataatgaccctttggtccttcgacggcgccagacctacttgtgctatgaggtggagcgcctggacaatggcacctgggtcctgatggaccagcacatgggctttctatgcaacgaggctaagaatcttctctgtggcttttacggccgccatgcggagctgcgcttcttggacctggttccttctttgcagttggacccggcccagatctacagggtcacttggttcatctcctggagcccctgcttctcctggggctgtgccggggaagtgcgtgcgttccttcaggagaacacacacgtgagactgcgcatcttcgctgcccgcatctatgattacgaccccctatataaggaggcgctgcaaatgctgcgggatgctggggcccaagtctccatcatgacctacgatgagtttgagtactgctgggacacctttgtgtaccgccagggatgtcccttccagccctgggatggactagaggagcacagccaagccctgagtgggaggctgcgggccattctccagaatcagggaaactgaaggatgggcctcagtctctaaggaaggcagagacctgggttgagcagcagaataaaagatcttcttccaagaaatgcaaacagaccgttcaccaccatctccagctgctcacagacaccagcaaagcaatgtgctcctgatcaagtagattttttaaaaatcagagtcaattaattttaattgaaaatttctcttatgttccaagtgtacaagagtaagattatgctcaatattcccagaatagttttcaatgtattaatgaagtgattaattggctccatatttagactaataaaacattaagaatcttccataattgtttccacaaacact

SEQ ID NO: 11 - DNA, 20 bp, artificial
  synthetic oligonucleotide primer
gagaagggacaagcacatgg

SEQ ID NO: 12 - DNA, 19 bp, artificial
  synthetic oligonucleotide primer
gaccctttggtccttcgac

SEQ ID NO: 13 - DNA, 20 bp, artificial
  synthetic oligonucleotide primer
agcgcttcagaaaagagtgg

SEQ ID NO: 14 - DNA, 20 bp, artificial
  synthetic oligonucleotide primer
acccaaacgtcagtcgaatc

SEQ ID NO: 15 - DNA, 18 bp, artificial
  synthetic oligonucleotide primer
ccgtttggacgcaaagat

SEQ ID NO: 16 - DNA, 19 bp, artificial
  synthetic oligonucleotide primer
ccgaggacccgaaggttac

SEQ ID NO: 17 - DNA, 18 bp, artificial
  synthetic oligonucleotide primer
agctgtggccagaagcac

SEQ ID NO: 18 - DNA, 25 bp, artificial
  synthetic oligonucleotide primer
gactttggttatcttcgcaataaga

SEQ ID NO: 19 - DNA, 22 bp, artificial
  synthetic oligonucleotide primer
gggaccttgttaacagtggagt

SEQ ID NO: 20 - DNA, 20 bp, artificial
  synthetic oligonucleotide primer
aagtagggcaactgggcttt

SEQ ID NO: 21 - DNA, 23 bp, artificial
  synthetic oligonucleotide primer
ttctaacacctggaatgtgatcc

SEQ ID NO: 22 - DNA, 18 bp, artificial
  synthetic oligonucleotide primer
cccatgactcccatgacc

SEQ ID NO: 23 - DNA, 20 bp, artificial
  synthetic oligonucleotide primer
tggatccatcaagtgtctgg

SEQ ID NO: 24 - DNA, 18 bp, artificial
  synthetic oligonucleotide primer
gcacagccccaggagaag

SEQ ID NO: 25 - DNA, 20 bp, artificial
  synthetic oligonucleotide primer
aagtttcgttccgatcgttg

SEQ ID NO: 26 - DNA, 20 bp, artificial
  synthetic oligonucleotide primer
cacatttctgcgtggttctc

SEQ ID NO: 27 - DNA, 21 bp, artificial
  synthetic oligonucleotide primer
ccaggtgatctggaaacactt

SEQ ID NO: 28 - DNA, 20 bp, artificial
  synthetic oligonucleotide primer
tccaacagtgctgaaattcg

SEQ ID NO: 29 - DNA, 18 bp, artificial
  synthetic oligonucleotide primer
cggaatgtttcggctgtt

SEQ ID NO: 30 - DNA, 20 bp, artificial
  synthetic oligonucleotide primer
aggtcccagtccgagatgta

SEQ ID NO: 31 - DNA, 21 bp, artificial
  synthetic oligonucleotide primer
ccaggtgggtagttgacaaaa

SEQ ID NO: 32 - DNA, 22 bp, artificial
  synthetic oligonucleotide primer
ggctgtacatgtcattgctgtc

SEQ ID NO: 33 - DNA, 24 bp, artificial
  synthetic oligonucleotide primer
tttactgtcttctagctgcaaacc

SEQ ID NO: 34 - DNA, 23 bp, artificial
  synthetic oligonucleotide primer
tttacaaccaagattcactgtgg

SEQ ID NO: 35 - DNA, 8 bp, artificial
  synthetic oligonucleotide probe
ctgggctg

SEQ ID NO: 36 - DNA, 8 bp, artificial
  synthetic oligonucleotide probe
cctggagc

SEQ ID NO: 37 - DNA, 8 bp, artificial
  synthetic oligonucleotide probe
ttgccttc

SEQ ID NO: 38 - DNA, 8 bp, artificial
  synthetic oligonucleotide probe
ggcaggag

SEQ ID NO: 39 - DNA, 8 bp, artificial
  synthetic oligonucleotide probe
gctgcctg

SEQ ID NO: 40 - DNA, 8 bp, artificial
  synthetic oligonucleotide probe
ccaggagg

SEQ ID NO: 41 - DNA, 8 bp, artificial
  synthetic oligonucleotide probe
tggctctg

SEQ ID NO: 42 - DNA, 8 bp, artificial
  synthetic oligonucleotide probe
ggaggaag

SEQ ID NO: 43 - DNA, 8 bp, artificial
  synthetic oligonucleotide probe
tgctggag

SEQ ID NO: 44 - DNA, 8 bp, artificial
  synthetic oligonucleotide probe
ctgctgcc

SEQ ID NO: 45 - DNA, 8 bp, artificial
  synthetic oligonucleotide probe
cctggaga

SEQ ID NO: 46 - DNA, 8 bp, artificial
  synthetic oligonucleotide probe
ggcaggag

SEQ ID NO: 47 - DNA, 21 bp, artificial
  synthetic single-stranded oligonucleotide
aaattctaatagataatgtga

SEQ ID NO: 48 - DNA, 30 bp, artificial
  synthetic oligonucleotide primer
gagctggaccttaggctccagaaaggacaa

SEQ ID NO: 49 - DNA, 27 bp, artificial
  synthetic oligonucleotide primer
gctggtgttgttgggcagtgctaggaa

SEQ ID NO: 50 - DNA, 29 bp, artificial
  synthetic oligonucleotide primer
ttctcttttcctatcctgagtagtggtaa

SEQ ID NO: 51 - DNA, 30 bp, artificial
  synthetic oligonucleotide primer
aaaggtgataaaagtgaatctgaggcataa

SEQ ID NO: 52 - DNA, 26 bp, artificial
  synthetic oligonucleotide primer
atgcccctcaacgttagcttcaccaa

SEQ ID NO: 53 - DNA, 24 bp, artificial
  synthetic oligonucleotide primer
ctggatgatgatgtttttgatgaa

SEQ ID NO: 54 - DNA, 25 bp, artificial
  synthetic oligonucleotide primer
acgaggaggagaacttctaccagca

SEQ ID NO: 55 - DNA, 25 bp, artificial
  synthetic oligonucleotide primer
tctcgtcgtccgggtcgcagatgaa

SEQ ID NO: 56 - DNA, 23 bp, artificial
  synthetic oligonucleotide primer
tgtgtgccaggttgccactctca

SEQ ID NO: 57 - DNA, 22 bp, artificial
  synthetic oligonucleotide primer
aagcccaagtactgcctgggga

SEQ ID NO: 58 - DNA, 21 bp, artificial
  synthetic oligonucleotide primer
cccagtaggcggagcagagga

SEQ ID NO: 59 - DNA, 20 bp, artificial
  synthetic oligonucleotide primer
agggagccgccaggaatgct

SEQ ID NO: 60 - DNA, 14 bp, artificial
  sample sequence demonstrating DNA repair pathway
atgtcattacatat

SEQ ID NO: 61 - DNA, 14 bp, artificial
  sample sequence demonstrating DNA repair pathway
atatgtaatgacat

SEQ ID NO: 62 - DNA, 14 bp, artificial
  sample sequence demonstrating DNA repair pathway
atgtuattauatat

SEQ ID NO: 63 - DNA, 14 bp, artificial
  sample sequence demonstrating DNA repair pathway
atatgtaatgauat

SEQ ID NO: 64 - DNA, 14 bp, artificial
  sample sequence demonstrating DNA repair pathway
atgtnattanatat

SEQ ID NO: 65 - DNA, 14 bp, artificial
  sample sequence demonstrating DNA repair pathway
atatgtaatganat

SEQ ID NO: 66 - DNA, 14 bp, artificial
  sample sequence demonstrating DNA repair pathway
atattattatatat

SEQ ID NO: 67 - DNA, 14 bp, artificial
  sample sequence demonstrating DNA repair pathway
atatataataatat

SEQ ID NO: 68 - protein, 429 aa, Homo sapiens
  MISC_FEATURE (372)..(429): amino acids present in an alternative form of A3B resulting from a frame-shift mutation
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGEAAGHSPESGKLKDGPQSLRKAETWVEQQNKRSSSKKCKQTVHHHLQLLTDTSKAMCS

(19) United States
(12) Patent Application Publication
(10) Pub. No.: US 2012/0252026 A1
(43) Pub. Date: Oct. 4, 2012

(54) CANCER BIOMARKER, DIAGNOSTIC METHODS, AND ASSAY REAGENTS

(76) Inventors: REUBEN S. HARRIS, St. Paul, MN (US); Michael Burns, Minneapolis, MN (US); Eric W. Refsland, Minneapolis, MN (US)

(21) Appl. No.: 13/437,484

(22) Filed: Apr. 2, 2012

Related U.S. Application Data
(60) Provisional application No. 61/470,954, filed on Apr. 1, 2011.

(51) Int. Cl. C12Q 1/68; B32B 1/02; C07H 21/00

(52) U.S. Cl. 435/6.12; 536/23.1; 428/34.1

(57) ABSTRACT

This disclosure describes APOBEC3B as a biomarker for certain cancers such as, for example, breast cancer. This disclosure therefore describes methods for detecting APOBEC3B in a biological sample. The methods generally include measuring expression of APOBEC3B in a biological sample obtained from a patient and identifying the patient as having or at risk for having cancer if the measured expression of APOBEC3B is greater than a predetermined reference level of expression. This disclosure also describes isolated polynucleotides that may be used as reagents in methods for detecting and/or measuring APOBEC3B expression.

### CROSS-REFERENCE TO RELATED APPLICATION

This application claims priority to U.S. Provisional Patent Application Ser. No. 61/470,954, filed Apr. 1, 2011.

### BACKGROUND

Spontaneous hydrolytic deamination of DNA cytosine to uracil (C-to-U) or methyl-cytosine to thymine (mC-to-T) is a well-established pro-mutagenic process, which, by DNA replication or misrepair, can result in C/G-to-T/A transition mutations (Lindahl, 1993 Nature 362:709-715; Krokan et al., 2002 Oncogene 21:8935-8948). However, the stochastic nature of this process is difficult to reconcile with the fact that strong C/G-to-T/A mutation biases are not observed in all human cancers, but only in substantive subsets (Sjöblom et al., 2006 Science 314:268-274; Berger et al., 2011 Nature 470:214-220). Also difficult to explain is the fact that spontaneous deamination of mC is 4.5-fold more efficient than that of C (Ehrlich et al., 1986 Biosci Rep 6:387-393), yet many human cancer C/G-to-T/A transition mutations occur in non-methylated dinucleotide motifs (i.e., outside of CpG motifs). Specific examples include breast cancer, brain cancer, and prostate cancer, among others where over half of the C/G-to-T/A transitions occur within non-CpG dinucleotide sequences (Ding et al., 2010 Nature 464:999-1005; Greenman et al., 2007 Nature 446:153-158; Sjoblom et al., 2006 Science 314:268-274; Stephens et al., 2009 Nature 462:1005-1010; Berger et al., 2011 Nature 470:214-220). These observations strongly indicate that an alternative mechanism underlies this C/G-to-T/A transition mutation bias.

### SUMMARY OF THE INVENTION

In one aspect, this disclosure describes a method that generally includes measuring expression of APOBEC3B in a biological sample obtained from a patient and identifying the patient as having or at risk for having cancer if the measured expression of APOBEC3B is greater than a predetermined reference level of expression. In some embodiments, the predetermined reference level of expression can include the level of APOBEC3B expression in a normal cell. In some embodiments, the cancer can include breast cancer.

In another aspect, this disclosure describes a method that generally includes measuring expression of APOBEC3B in a biological sample obtained from a patient and determining a therapeutic parameter if the measured expression of APOBEC3B is greater than a predetermined reference level of expression, wherein the therapeutic parameter comprises a probability of outcome, likelihood of recurrence, likelihood of therapy resistance, or any combination thereof.

In each of the aspects summarized above, measuring expression of APOBEC3B can include quantitative PCR. In some of these embodiments, the quantitative PCR can include annealing a polynucleotide from the biological sample with a first primer that anneals to a first strand of a polynucleotide that encodes APOBEC3B and a second primer that anneals to a second strand of a polynucleotide that encodes APOBEC3B. In some of these embodiments, the first primer can include the nucleotide sequence of SEQ ID NO:12 and the second primer can include the nucleotide sequence of SEQ ID NO:24. In some embodiments, the quantitative PCR can further include detecting the polynucleotide sequence amplified using the primers by annealing a labeled probe to at least a portion of the amplified polynucleotide. In some embodiments, the probe can include the polynucleotide sequence of SEQ ID NO:36.

In another aspect, this disclosure describes certain isolated polynucleotides. In some embodiments, the isolated polynucleotide includes the nucleotide sequence of SEQ ID NO:12. In other embodiments, the isolated polynucleotide includes the nucleotide sequence of SEQ ID NO:24. In some embodiments, the isolated polynucleotides may be a primer.

In another aspect, this disclosure describes kits that can include one or more isolated polynucleotides as described herein. The kits—and the polynucleotides included therein—may be useful for detecting and/or measuring expression of certain A3 family proteins including, for example, APOBEC3B.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. A3 mRNA levels in the indicated cell lines presented relative to A3B mRNA levels in MCF10A, set to 1 for comparison.

FIG. 2. a, A3B mRNA levels in reduction mammoplasty samples (triangles, n=28) and matched sets of breast tumor in comparison to adjacent or contralateral normal tissue (circles and squares, respectively, n=52). Median A3B levels in each group are 0.024, 0.15, and 0.043, relative to the constitutive housekeeping gene TATA-Binding Protein, TBP (s.d. shown unless smaller than the data point). Data are arranged from lowest to highest A3B expression level, except each matched normal data point is presented directly below the corresponding tumor data point. b, A3G levels in the same samples as in (a). Median A3G levels in each group are 0.84, 1.1, and 1.1, relative to the constitutive housekeeping gene TBP.

FIG. 3. a, A3B levels in the indicated breast cancer (circles, n=44) and non-cancerous (squares, n=2) cell lines. Each data point is the mean A3B level of three qPCR reactions presented relative to mRNA levels of the constitutive housekeeping gene TBP (s.d. shown unless smaller than the data point). Data are arranged from lowest to highest A3B expression level. Arrows highlight cell lines used in mechanistic studies in this paper. b, e, h, A3B-eGFP co-localizes with nuclear DNA (DAPI-stained), whereas A3F-eGFP is cytoplasmic, in the indicated live breast cancer cell lines. c, f, i, A3B mRNA levels in the indicated breast cancer cell lines stably transduced with shA3B or shControl lentiviruses. d, g, j, DNA C-to-U activity in nuclear extracts of the indicated breast cancer cell lines transduced as in (b).

FIG. 4. Full expression profiles for APOBEC family members in 10 randomly selected sets of matched breast tumor and normal tissue. Ten representative breast tumor samples and the matched normal control tissues were used to synthesize cDNA for qPCR analyses of the full human APOBEC repertoire. Each data point is the mean mRNA level of three qPCR reactions presented relative to mRNA levels of the constitutive housekeeping gene TBP (s.d. shown as a bar unless smaller than the data point). P-values are indicated for each family member (except A2 and A4, where the majority of samples had no detectable mRNA for these targets). A3B emerges as the only differentially expressed family member in tumor versus matched normal tissues. Samples are presented in order of an arbitrarily assigned patient number. The A3B and A3G data are also presented in FIG. 2 in the context of the full patient tissue expression dataset.

FIG. 5. Full expression profiles for APOBEC family members in select cell lines of mammary origin. The indicated cell lines were used to generate cDNA for pPCR analyses of the full human APOBEC repertoire. Each data point is mean mRNA level of three qPCR reactions presented relative to mRNA levels of the constitutive housekeeping gene TBP (s.d. shown as a bar unless smaller than the data point). The A3B data are also presented in FIG. 3a in the context of the full panel of normal and breast cancer cell lines.

FIG. 6. APOBEC3B is active in the nuclear protein fraction of multiple breast cancer cell lines. a, A3 mRNA levels in the indicated breast cancer cell lines. Each column is mean+/−s.d. of three qPCR reactions presented relative to mRNA levels of the constitutive housekeeping gene TBP. Grey and black bars represent expression data from cells stably transduced with shControl or shA3B lentivirus, respectively. b, A3B-dependent DNA deaminase activity in the nuclear (Nuc) and cytoplasmic (Cyt) fractions obtained from the cell lines in (a). The fractionation was cleaner in MDA-MB-453 and MDA-MB-468 lines than HCC1569, but all detectable deaminase activity was still dependent on A3B. c, Immunoblots showing the distribution of histone H3, a nuclear protein, and tubulin, a cytoplasmic protein, in the protein preparations used in (b) to confirm efficient sub-cellular fractionation.

FIG. 7. APOBEC3B-dependent uracils in breast cancer genomic DNA. a, Schematic of the workflow for genomic uracil quantification by HPLC-MS. b, A3B mRNA levels in the indicated breast cancer cell lines stably transduced with shControl or shA3B lentiviruses. c, Steady-state genomic uracil loads per mega-basepair (Mbp) in the indicated breast cancer cell lines expressing shControl or shA3B constructs.

FIG. 8. APOBEC3B-dependent hypermutation in breast cancer genomic DNA. a, Workflow for TK fluctuation analysis. b, A3B mRNA levels in representative TK^{positive} MDA-MB-453 subclones. c, Histogram depicting the median frequency of TK^{minus} mutants occurring during clonal outgrowth of MDA-MB-453 subclones expressing shControl or shA3B constructs. d, Agarose gel analysis of 3D-PCR amplicons obtained using primers specific for the indicated target genes and genomic DNA prepared from HCC1569 cells expressing shControl or shA3B constructs. The denaturation temperature range is indicated above each gel. e, Pie charts depicting the C/G-to-T/A mutation load in 3D-PCR products after cloning and sequencing (n≧40 per condition). Charts align with target genes labeled in (d). f, Hypermutable state model. A3B over-expression causes elevated levels of ongoing mutation, which may contribute to all stages of tumor development.

FIG. 9. A model of the balance between APOBEC3B DNA mutation and the cellular repair machinery. a, In normal tissues, A3B is not expressed or expressed at very low levels. In the rare event that A3B does deaminase genomic C-to-U, the DNA repair machinery rapidly repairs the DNA and thereby prevents the mutation. b, In many cancers, e.g., breast cancer, A3B is massively overexpressed and highly active. Some, but not all, of the uracil lesions it generates can be repaired by the DNA repair pathways. This balance is tightly controlled at the level of base excision repair by UNG2. Sample sequences (SEQ ID NOs: 60-67) are used in the schematic.

### DETAILED DESCRIPTION OF ILLUSTRATIVE EMBODIMENTS

Multiple mutations may be required for cancer development and many cancers, including breast cancer, can harbor few causative mutations but numerous hitch-hikers. Here we demonstrate that the DNA cytosine deaminase APOBEC3B is an endogenous source of mutation in breast cancer. We show that APOBEC3B is overexpressed in many primary breast tumors (24/37) and available breast cancer cell lines (34/44). APOBEC3B may be found in the cell nucleus and it has DNA C-to-U activity in breast cancer cell line extracts. This pro-mutagenic activity broadly impacts the integrity of breast cancer genomic DNA because it can create an abundance of uracil lesions that can effectively overwhelm normal base excision repair processes. For instance, prolonged inhibition of the uracil-specific base excision repair protein UNG2 in APOBEC3B-overexpressing breast cancer cells can lead to error catastrophe and massive cell death. Thus, APOBEC3B-catalyzed DNA deamination is a source of DNA lesions in breast cancer, can account for hallmark C/G-to-T/A transitions and, depending on DNA repair pathway usage and fidelity, possibly also for other types of base substitution mutations and larger-scale chromosomal aberrations. Consequently, APOBEC3B may be a diagnostic marker for cancer including, for example, breast cancer.

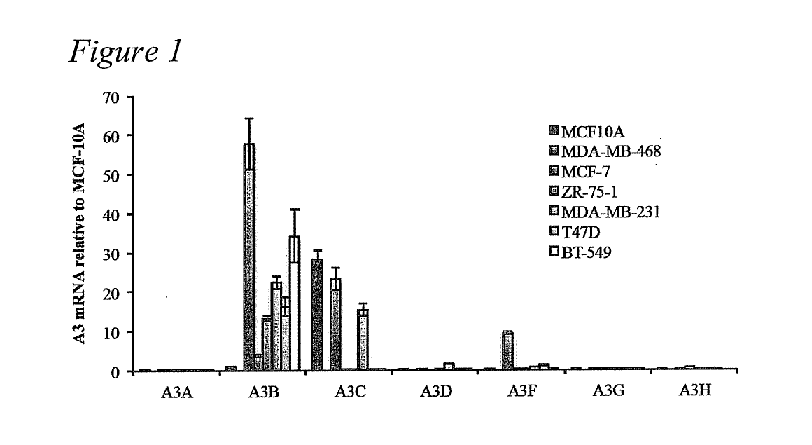

Our initial survey used highly specific and quantitative reverse transcription PCR (qRT-PCR) assays to examine APOBEC3 (A3) family mRNA expression levels in six breast cancer cell lines. In comparison to MCF10A, considered an immortalized breast epithelial control line, all six lines expressed elevated levels of APOBEC3B (A3B), a member of A3 family: MDA-MB-468 (58-fold increase), MCF-7 (4-fold increase), ZR-75-1 (13-fold increase), MDA-MB-231 (22-fold increase), T47D (16-fold increase), and BT-549 (34-fold increase) (FIG. 1). Levels of other A3 family mRNAs were similar to those found in MCF10A (A3C), undetectable (A3A, A3G, A3H), or only weakly expressed (A3D, A3F).

“Expressed” and variations thereof refer to the conversion of genetic information in a nucleotide sequence to a gene product. Expression of a nucleotide sequence (e.g., a gene) may be measured and/or described with reference to (a) transcription of DNA to mRNA, (b) translation of mRNA to protein, (c) post-translational steps (e.g., modification of the primary amino acid sequence; addition of a phosphate, carbohydrate, a lipid, a nucleotide, or other moiety to the protein; assembly of subunits; insertion of a membrane-associated protein into a biological membrane; and the like), or any combination of the foregoing. In some cases, therefore, expression of APOBEC3B can refer to the production of an APOBEC3B polypeptide such as, for example, a polypeptide that includes the amino acid sequence of, for example, SEQ ID NO:9 or another known polymorph or variant thereof. In other contexts, expression of APOBEC3B can refer to the production of a polynucleotide (e.g., an mRNA) that encodes such an APOBEC3B polypeptide such as, for example, the polynucleotide sequence of SEQ ID NO:10.

As used herein, an “APOBEC3B polypeptide” refers to a polypeptide that includes the amino acid sequence of SEQ ID NO:9 or any polypeptide that is structurally similar to SEQ ID NO:9. As used herein, a polypeptide is “structurally similar” to a SEQ ID NO:9 if the amino acid sequence of the polypeptide possesses a specified amount of identity compared to the reference polypeptide. Structural similarity of two polypeptides can be determined by aligning the residues of the two polypeptides (for example, a candidate polypeptide and the polypeptide of SEQ ID NO:9) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate polypeptide is the polypeptide being compared to SEQ ID NO:9. A candidate polypeptide can be isolated, for example, from an animal, or can be produced using recombinant techniques, or chemically or enzymatically synthesized.

A pair-wise comparison analysis of amino acid sequences can be carried out using the BESTFIT algorithm in the GCG package (version 10.2, Madison Wis.). Alternatively, polypeptides may be compared using the Blastp program of the BLAST 2 search algorithm, as described by Tatiana et al., (FEMS Microbiol Lett, 174, 247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a polypeptide of the invention may be selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH2. Likewise, biologically active analogs of a polypeptide containing deletions or additions of one or more contiguous or noncontiguous amino acids that do not eliminate a functional activity of the polypeptide are also contemplated.

For example, known APOBEC3B variants include amino acid substitutions in the amino acid sequence of SEQ ID NO:9. Exemplary substitutions include, for example, a lysine to glutamic acid substitution at position 62 (K62E), a proline to leucine substitution at position 98 (P98L), a serine to alanine substitution at position 109 (S109A), a threonine to lysine substitution at position 146 (T146K), a tyrosine to histidine substitution at position 191 (Y191 H), an arginine to histidine substitution at position 212 (R212H), a glutamic acid to aspartic acid substitution at position 342 (E342D), a tyrosine to histidine substitution at position 343 (Y343H), and an arginine to histidine substitution at position 351 (R351H). In addition, another known human APOBEC3B variant (SEQ ID NO:68) includes a substitution of the C-terminal eleven amino acids 372-382 with a 58 amino acid sequence.

An APOBEC3B polypeptide therefore can include a polypeptide with at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to the reference amino acid sequence.

An APOBEC3B polypeptide therefore can include a polypeptide with at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the reference amino acid sequence.

As used herein, a “polynucleotide that encodes an APOBEC3B polypeptide” refers to any polynucleotide that encodes the amino acid sequence of SEQ ID NO:9 or a polynucleotide that encodes any of the APOBEC3B polypeptide variant (e.g., those variants described above). Certain known single-nucleotide polymorphisms (SNPs) result in the amino acid variants described above. Other SNPs may not change the amino acid sequence of the encoded APOBEC3B polypeptide. Exemplary SNPs can include, for example, the following: C123T (rs113972364), A238G (rs2076109), C252T (rs17000717), C347T (rs59708943), T379G (rs17000697), T411C (rs2076111), C491A (rs5995649), C603G (rs11705335), T625C (rs61730556), T672C (rs35949382), G689A (rs113792154), C999T (rs1065184), G1080C (rs6001357), T1081C (rs1133004), G1106A (rs1053813), T1164TG (rs34383929), C1219G (rsl 11603647), and T1459C (rs1804930), where in the format X###Y, X is the consensus nucleotide base, ### is the position of the base in SEQ ID NO:10, and Y is the substituted base at that position. The NCBI dbSNP reference number for each SNP is provided in parentheses.

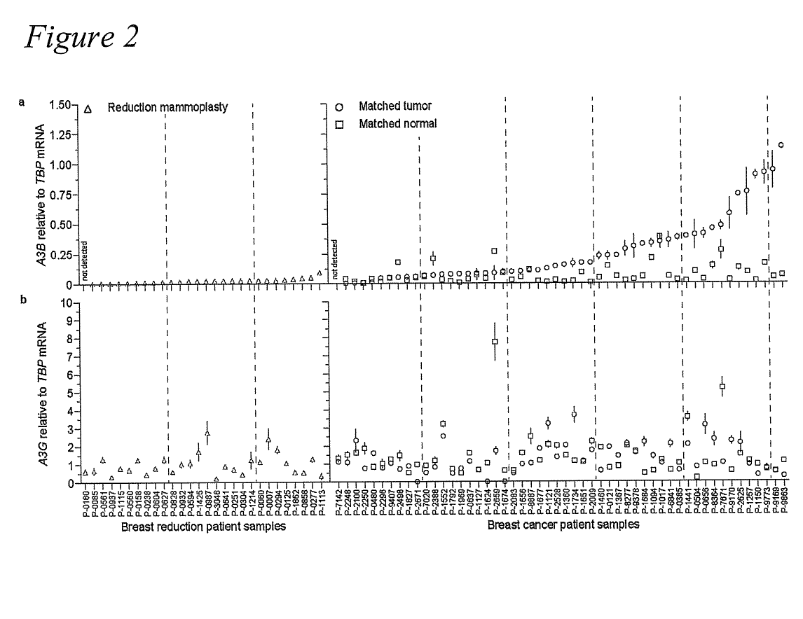

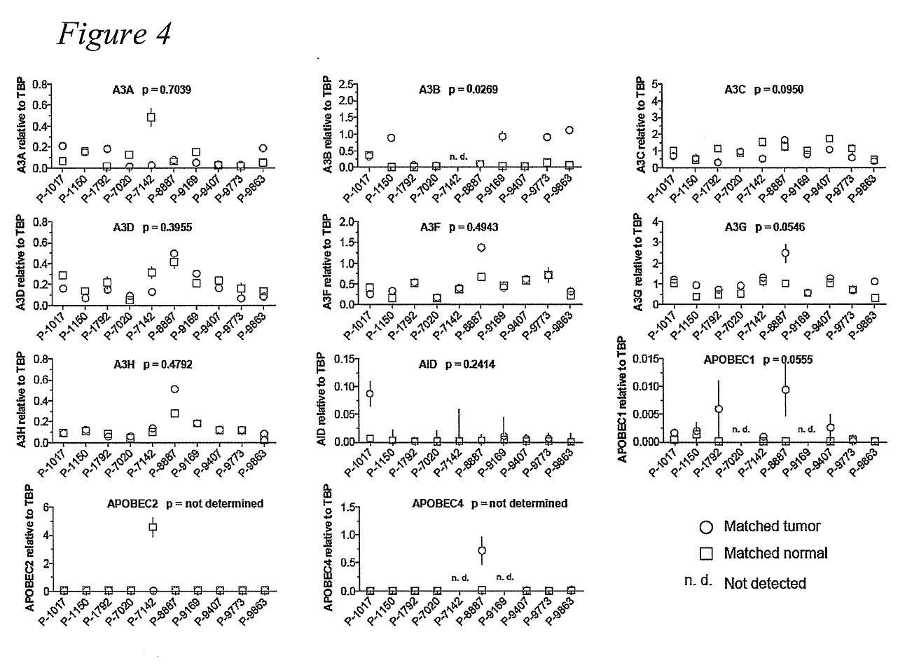

To extend these observations to primary tissues and exclude potential artifacts of cell culture, we quantified A3B mRNA levels in 37 patient-matched normal and tumor breast tissue specimens. Tumor tissues were derived from flash-frozen lumpectomies or mastectomies and normal tissues were procured simultaneously from an area adjacent to the tumor or from the contralateral breast. All data were normalized relative to A3B levels in each patient's corresponding normal tissue. Approximately two-thirds of all tumors had at least 2-fold A3B mRNA overexpression (24/37), with numerous tumors showing levels 10-fold above those in the matched normal tissue (10/37) (FIG. 2a). We note that these values are highly significant (p=0.000003) and quite likely underestimates because each specimen contains numerous non-tumor non-A3B expressing cell types. As additional controls, A3B levels in breast tissues procured during reduction surgeries (presumably normal) were not significantly different than the tumor-matched normal samples (FIG. 2a). Moreover, expression levels of the six other A3s showed no significant correlation (FIG. 2b, FIG. 4).

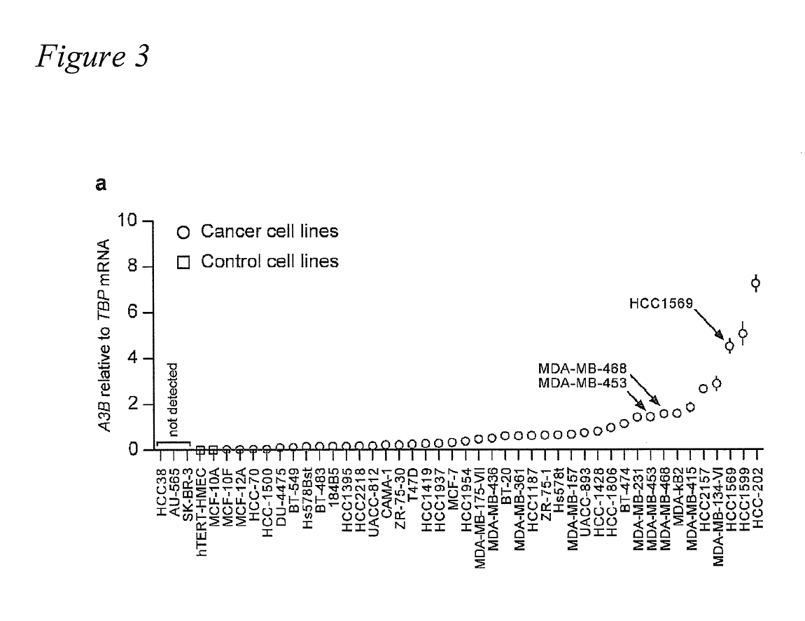

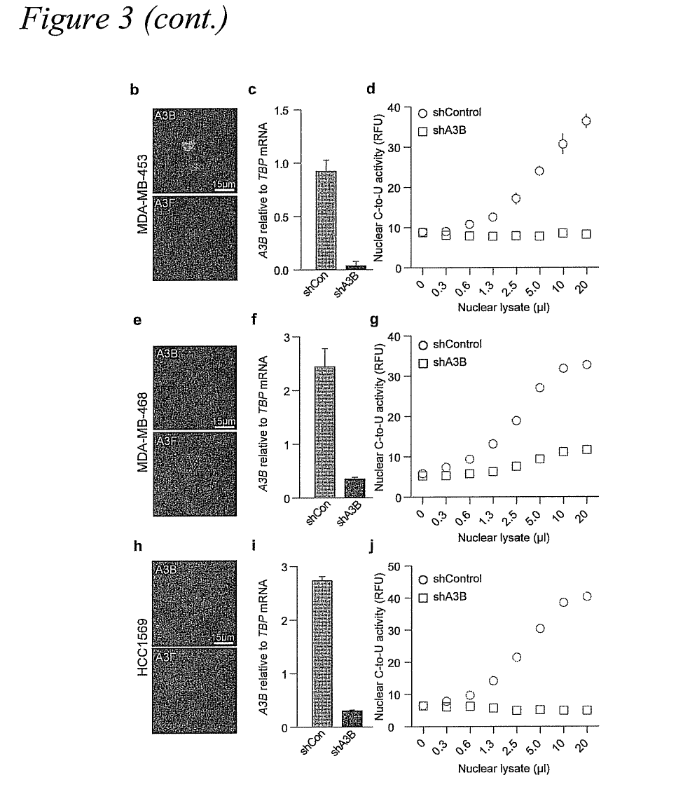

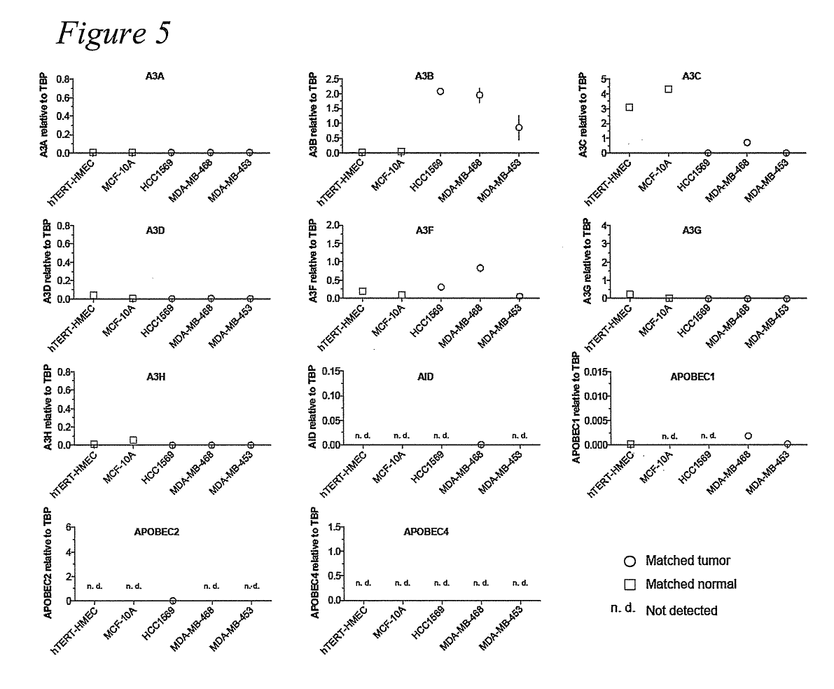

To distinguish between paracrine (trans) and autocrine (cis) mechanisms for A3B over-expression and establish systems for molecular studies, we analyzed mRNA levels in 44 breast cancer cell lines. A3B was over-expressed in nearly all of the lines, with levels ranging from two-fold to nearly 500-fold higher than control lines MCF10A or telomerase-immortalized mammary epithelial cells (FIG. 3a). Consistent with underestimates in primary tissues above, 33/44 lines had A3B levels ≧10-fold above controls. MDA-MB-453, HCC-1569, and MDA-MB-468, representative lines used below, showed 97-fold, 300-fold, and 105-fold over-expression, respectively (FIG. 3a, FIG. 5). At the opposite end of the expression spectrum, HCC38, AU-565, and SK-BR-3 had no detectable A3B, consistent with a circulating A3B deletion allele (Kidd et al., 2007 PLoS Genet 3:e63; Komatsu et al., 2008 Int. J. Oncology 33:261-270). Taken together, data from dozens of independent tumors and cell lines demonstrate that A3B mRNA is over-expressed strongly in breast cancer. Over-expression may involve at least one upstream cell autonomous genetic event because expression levels are low in normal tissues, high in most tumors, and heritably high in the majority of breast cancer cell lines—even among those that were cloned or subcloned—grown under many different conditions.

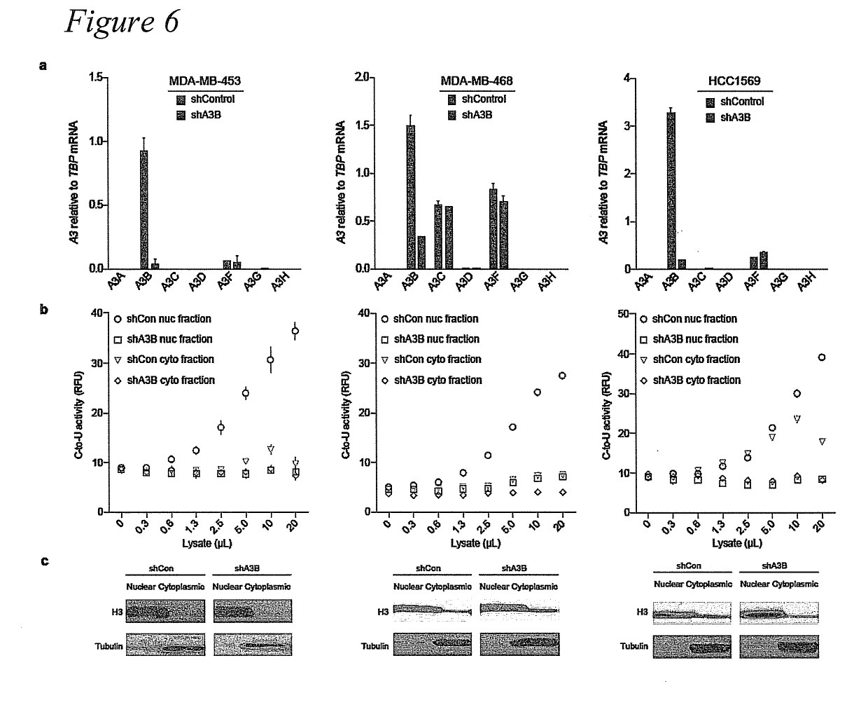

A3B-eGFP can concentrate in the nuclei of transiently transfected HeLa or HEK293T cells. To determine whether this is also the case in breast cancer cell lines, an A3B-eGFP expression construct was transfected into MDA-MB-453, MDA-MB-468, and HCC1569 cells and images were taken at 48 hours post-transfection by live cell fluorescent microscopy. Live cell images show A3B-eGFP nuclear localization (FIGS. 3b, 3e, and 3h). In contrast, A3F-eGFP localized to the cytoplasm (FIGS. 3b, 3e, and 3h). Similar data were obtained for A3B-HA and A3F-HA localization in fixed breast cancer cell lines (not shown). Thus, A3B is precariously close to genomic DNA and, although expressed in some breast cancer cell lines (e.g., FIG. 5 and FIG. 6), endogenous A3F is less likely to be genotoxic due to its cytoplasmic localization.

We next asked whether breast cancer cell nuclei harbor DNA C-to-U activity and whether this activity is due to endogenous A3B. These experiments were done by transducing breast cancer cell lines with lentiviruses that express short hairpin RNA to A3B (shA3B) or scrambled control RNA (shControl), fractionating the nuclear and cytoplasmic proteins, and using the resulting extracts to perform DNA C-to-U activity assays with fluorescently labeled DNA substrates. A3B mRNA levels declined by >85% in knockdown cells (FIGS. 3c, 3f, and 3i). Knockdown specificity was evidenced by no reduction in A3C or A3F mRNA (FIG. 6). High levels of A3B-dependent DNA C-to-U activity were only found in nuclear protein fractions (FIGS. 3d, 3g, and 3h and FIG. 6).

Moreover, whole cell protein extracts contained high levels of A3B-dependent DNA C-to-U activity, and this activity clearly tracked with nuclear and not cytoplasmic fractions (FIGS. 6b and 6c). This direct correlation between A3B mRNA levels and deaminase levels demonstrate that endogenous A3B is responsible for most, if not all, DNA C-to-U activity in the nuclear compartment of several breast cancer cell lines.

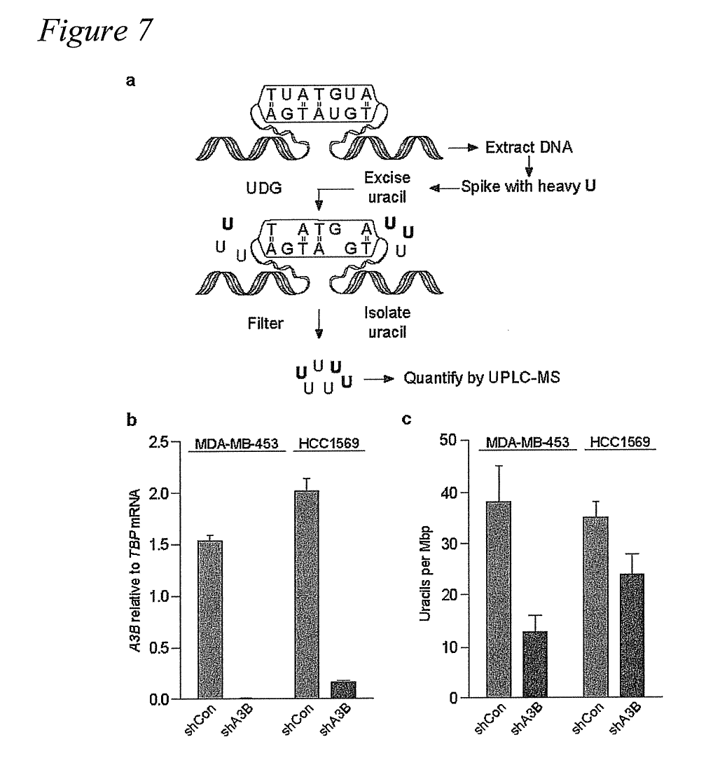

To ask whether endogenous A3B causes uracil lesions in the genomic DNA of breast cancer cells, we quantified genomic uracil levels in MDA-MB-453 and HCC 1569 (FIG. 7a). After shA3B or shControl transduction and 3 weeks in culture, genomic DNA was prepared, spiked with heavy-labeled uracil as an internal standard, treated with uracil DNA glycosylase, fractionated to liberate uracil from larger molecules, and subjected to separation by ultra-performance liquid chromatography and detection by mass spectrometry. A3B was depleted >90% from each cell line with greater knockdown in MDA-MB-453 than HCC1569 (FIG. 7b). This trend was reflected in the uracil quantification data with A3B knockdown causing 3-fold less genomic uracil in MDA-MB-453 and <2-fold in HCC1569 (FIG. 7c). Although these relative differences may seem modest, 20 uracils and 10 uracils per Mbp, respectively, this translates to 60,000 and 30,000 A3B-dependent uracils per haploid genome. Moreover, these steady state values may underestimate the actual number of A3B catalyzed pro-mutagenic lesions because mismatch repair and several base excision repair pathways undoubtedly counteract this DNA damage.

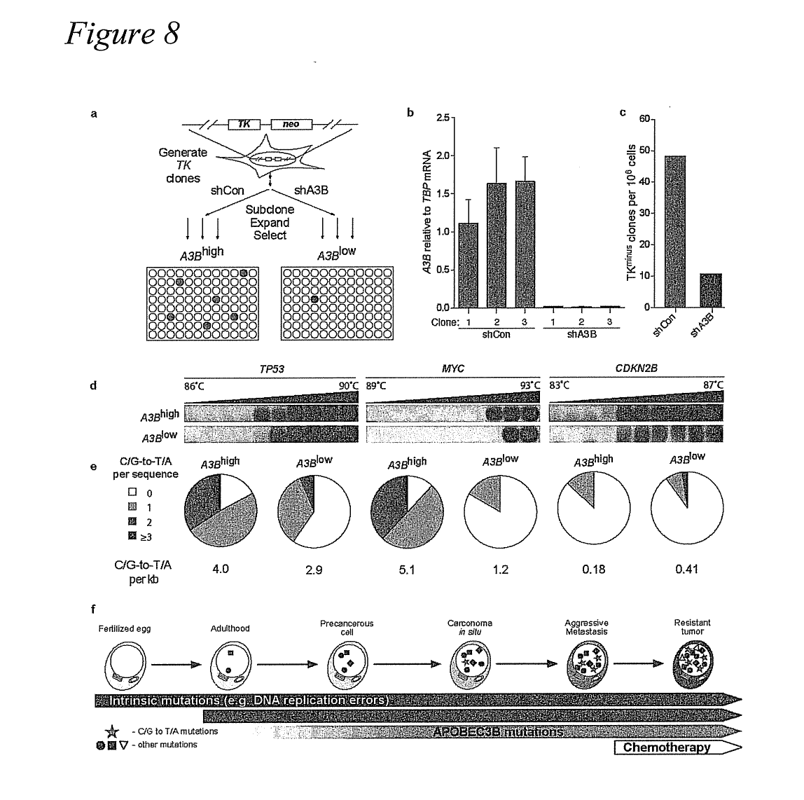

To determine whether elevated uracil loads lead to mutations, a thymidine kinase-positive (TK^{plus}) to thymidine-negative (TK^{minus}) fluctuation analysis was used to determine mutation frequencies (FIG. 8a). MDA-MB-453 cells were engineered to express TK, which confers sensitivity to the drug ganciclovir. This TK^{plus} line was transduced with shA3B or shControl constructs (FIG. 8b). Limiting dilution was used to generate single cell A3B^{low} and A3B^{high} sub-clones, respectively. Expanded sub-clones were subjected to ganciclovir, and mutant TK^{minus} cells were allowed several weeks to form visible colonies. Finally, colony counts enabled the median mutation frequencies to be determined, which revealed that cells over-expressing endogenous A3B can accumulate five-fold more mutations (FIG. 8c).

Finally, 3D-PCR (Stenglein et al., 2010 Nat Struct Mol Biol 17:222-229; Suspène et al., 2011 Proc Natl Acad Sci USA 108, 4858-4863) was used to ask whether C/G-to-T/A transition mutations accumulate at three genomic loci in A3B^{low} and A3B^{high} pools of HCC1569 cells. This technique enables qualitative estimates of genomic hypermutation within a population of cells because DNA sequences with higher T/A content amplify at lower denaturation temperatures than parental sequences. Lower temperature amplicons were detected for TP53 and c-MYC, but not CDKN2B (FIG. 8d). Individual low temperature amplicons were cloned and sequenced, and higher levels of C/G-to-T/A transition mutations were observed in A3B^{high} in comparison to A3B^{low} genomic DNA samples (FIG. 8e). Other types of base substitution mutations were rare and did not correlate with A3B levels (not shown). Some mutations were still evident in the A3B^{low} samples, which may be due to residual deaminase activity and/or to the amplification power of 3D-PCR. Nevertheless, taken together, activity assays, uracil quantification experiments, TK fluctuation tests, 3D-PCR analysis, and sequencing analysis strongly support the conclusion that endogenous A3B deaminates the genomic DNA of breast cancer cells leading to C/G-to-T/A hypermutation (FIG. 8f).

Here, we provide the first compelling evidence for the active involvement of the DNA deaminase A3B in human cancer, specifically breast cancer. Our work exposes the likelihood that other human cancers—particularly those such as brain and prostate cancers that manifest elevated C/G-to-T/A mutation levels—may involve over-expression of one or more DNA deaminase family members. Overall, many types of cancer may involve enzyme-catalyzed DNA deamination that can elevate mutation rates and thereby supply the genetic variability that can support cancer development, metastasis, and/or therapy resistance.

Without being bound by any particular theory, a delicate balance may exist in human cells between the beneficial innate immune functions afforded by the A3 proteins and minimizing potentially harmful consequences of genomic hypermutation. For example, precancerous cells may accrue at least one prior mutation to induce A3B and/or inactivate a repair path before A3B DNA deaminase activity compromises the integrity of the genome.

Hints of A3 over-expression have been reported for breast (Harris et al., 2002 Mol Cell 10:1247-1253) and other cancers (Harris et al., 2002 Mol Cell 10:1247-1253; Xu et al., 2007 Hepatology 46:1810-1820). The high level of identity between A3G, A3B, and several other A3 cDNAs, however, has prevented unambiguously implication of any particular A3 family member. Here, we use highly specific quantitative RT-PCR assays (Refsland et al., 2010 Nucleic Acids Res 38:4274-4284) to survey breast cancer tissues and cell lines for expression of each of the seven APOBEC3 genes. Significant A3B overexpression is clearly evident in 24/37 primary breast tumors and 33/44 available breast cancer cell lines. A3B overexpression manifests at the protein level as well, with high levels of A3B-dependent DNA C-to-U activity evident in the nuclear compartment of breast cancer cell lines. A3B-specific snRNA knockdown in several breast cancer cell lines demonstrated a genomic DNA deamination mechanism involving excessive levels of uracil, elevated mutation rates, and extensive hypermutation.

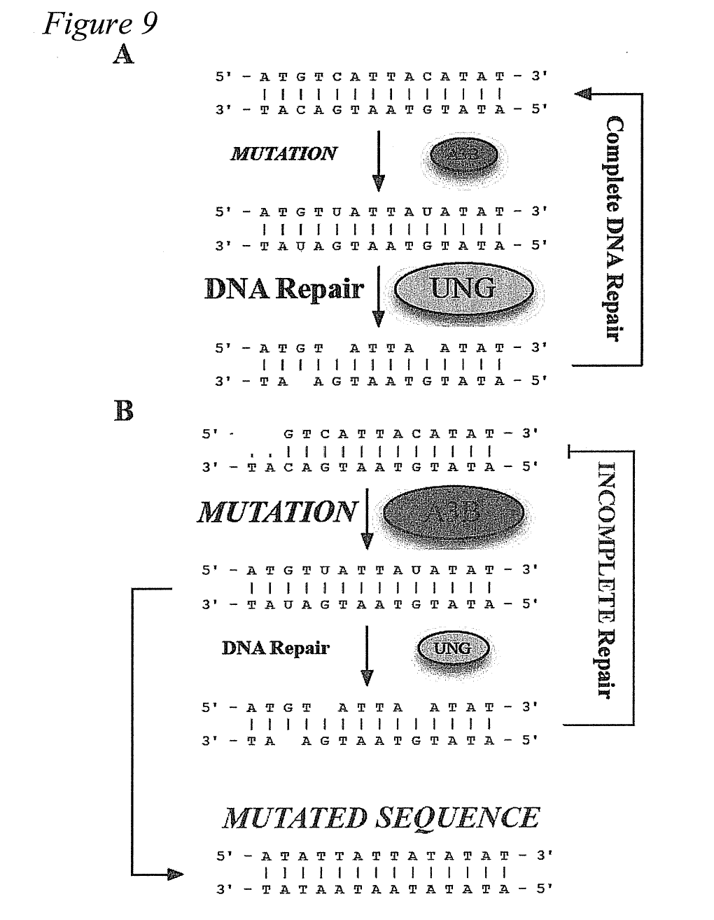

Thus, the genomic DNA of certain cancer cells (e.g., breast cancer cell lines) may be susceptible to elevated levels of genomic DNA deamination. Inhibiting uracil excision repair pathways can tip the balance afforded by A3 proteins from repair—and, therefore, cell viability—to a failure to repair and, therefore, error catastrophe and cell death. These data combine to suggest a model in which many A3B-dependent lesions escape repair during breast cancer development, underlying the observed C/G-to-T/A mutation bias and possibly also the frequently observed chromosomal aberrations (FIG. 9; Ding et al., 2010 Nature 464:999-1005; Greenman et al., 2007 Nature 446:153-158; Sjoblom et al., 2006 Science 314:268-274; Stephens et al., 2009 Nature 462:1005-1010; Berger et al., 2011 Nature 470:214-220). Taken together with tumor C/G-to-T/A mutation biases apparent in deep-sequencing datasets, we suggest that A3B contributes to a hypermutable state and to ongoing breast cancer evolution in vivo (FIG. 8f). The same mechanism may be involved in other cancers with strikingly similar hypermutation biases.

Since multiple mutations are required for a normal cell to progress to a tumorigenic state, elevated mutation rates have long been suspected in carcinogenesis (Loeb, 1991 Cancer Res 51:3075-3079). Overt evidence for a mutator state is provided by general knowledge that transient exposures to UV and other environmental mutagens significantly increase the risk of cancer. Genetics evidence in favor of the mutator hypothesis is best exemplified by hereditary nonpolyposis colorectal cancer, which is due to inherited defects in mismatch repair (Kolodner, 2000 Nature 407:687, 689; Lengauer et al., 1998 Nature 396:643-649). Such defects are typically loss of function mutations that cause a passive failure to repair spontaneous lesions in genomic DNA.

Thus, APOBEC3B may be used as a diagnostic marker for certain cancers. APOBEC3B quantification also may assist with prognostic determinations such as the probabilities of outcome, recurrence, therapy resistance, etc. We observed A3B overexpression in nearly two-thirds of all breast tumors analyzed (24/37; FIG. 2). In comparison, the current prevalence of p53 tumor suppressor mutations in breast cancer is 25%, amplification of the HER2 oncogene is approximately 25%, and inactivation of the BRCA1/2 tumor suppressors occurs at a combined rate 5-10%. Our data further suggest, however, that the utility of ABOBEC3B as a diagnostic and/or prognostic may not be limited to breast cancer. Rather, the data we have generated using breast cancer cells is indicative of a more general phenomenon. Thus, APOBEC3B can have utility as a diagnostic and/or prognostic marker for any cancer that involves C/G-to-T/A hypermutation. Such cancers include, for example, ovarian cancer, prostate cancer, brain cancer, and head/neck cancer.

Thus, in one aspect, this disclosure describes a cancer diagnostic method. Generally, the method includes measuring expression of APOBEC3B in a biological sample obtained from a patient and identifying the patient as having or at risk for having cancer if the measured expression of APOBEC3B is greater than a predetermined reference level of expression. In some embodiments, the predetermined reference level of expression can be the level of APOBEC3B expression in a normal cell.

In another aspect, this disclosure describes a method of determining the prognosis of a cancer patient. Generally, the method includes measuring expression of APOBEC3B in a biological sample obtained from a patient and determining the patient's prognosis based, at least in part, on whether the measured expression of APOBEC3B is greater than a predetermined reference level of expression. The prognosis may be provided in the form of a therapeutic parameter such as, for example, a probability of outcome, the likelihood of recurrence, the likelihood of therapy resistance, or any combination thereof. Here again, in some embodiments, the predetermined reference level of expression can be the level of APOBEC3B expression in a normal cell.

For both the diagnostic method and the prognostic method, APOBEC3B can serve as a diagnostic or prognostic biomarker for any cancer that involves C/G-to-T/A hypermutation such as, for example, breast cancer, ovarian cancer, prostate cancer, brain cancer, and head/neck cancer. In particular embodiments, the cancer can include breast cancer.

Also for both the diagnostic method and the prognostic method, measuring expression of APOBEC3B can include quantitative PCR. Quantitative PCR may be performed using routine methods using primers designed to anneal under PCR conditions to a polynucleotide that encodes at least a portion of APOBEC3B (e.g., SEQ ID NO:10), so that the detection of a polynucleotide amplified using the primers indicates the presence of an APOBEC3B-encoding polynucleotide in the biological sample being examined. In some embodiments, a suitable PCR primer can include a first primer and a second primer. A suitable first primer can include, for example, the nucleotide sequence of SEQ ID NO:12. A suitable second primer can include, for example, the nucleotide sequence of SEQ ID NO:24.

Measuring expression of APOBEC3B also can include labeling the amplified polynucleotide to assist in its detection and/or quantification. In some embodiments, the amplified polynucleotide can be labeled using a labeled probe that selectively binds to at least a portion of the polynucleotide that encodes APOBEC3B such as, for example, at least a portion of the polynucleotide amplified using the PCR primer pair. For example, if a polynucleotide is amplified using a first primer that includes the nucleotide sequence of SEQ ID NO:12 and a second primer that includes the nucleotide sequence of SEQ ID NO:24, one exemplary probe can include the nucleotide sequence of SEQ ID NO:36.

The increase in APOBEC3B expression that can indicate that a patient has or is at risk of having cancer, or that can be indicative of a particular prognosis, can be any increase in expression that reflects at least a two-fold APOBEC3B expression compared to normal cell. Thus, the increase in APOBEC3B expression can be, for example, at least two-fold, at least three-fold, at least four-fold, at least five-fold, at least six-fold, at least seven-fold, at least eight-fold, at least nine-fold, at least 10-fold, at least 20-fold, at least 25-fold, at least 50-fold, at least 100-fold, at least 150-fold, at least 200-fold, at least 250-fold, at least 300-fold, at least 350-fold, at least 400-fold, at least 450-fold, or at least 500-fold of APOBEC3B expression observed in a normal cell. For example, in specific embodiments illustrated in FIG. 3, cancer cells can exhibit a 97-fold increase (MDA-MB-453), a 105-fold increase (MDA-MB-468), or a 300-fold increase (HCC1569) in APOBEC3B expression compared to APOBEC3B expression in a normal cell.

In yet another aspect, this disclosure describes a kit for detecting expression of an A3 family polypeptide such as, for example, APOBEC3B. In some embodiments, the kit can contain reagents that can be used to detect and/or measure levels of an isolated polynucleotide that encodes the A3 family polypeptide (e.g., mRNA) obtained from a biological sample. The kit also may include a reagent that specifically binds, anneals, or otherwise labels the amplified polynucleotide for detection and/or quantification. In some embodiments, therefore, the kit reagents can include one or more primers and/or probes provided in Table 2, below. In one particular embodiment, the kit may be designed to detect and/or measure expression of APOBEC3B. In such an embodiment, the kit can include primers that may be used to amplify a polynucleotide obtained from a biological sample that encodes APOBEC3B. Exemplary suitable primers can include, for example, the nucleotide sequence of SEQ ID NO:12 or SEQ ID NO:24. A kit for detecting expression of APOBEC3B also can include a probe for detecting the amplified portion of the polynucleotide that encodes APOBEC3B. One suitable exemplary probe can include the nucleotide sequence of SEQ ID NO:36. In some embodiments, the probe can include a label (e.g., a radiolabel, a fluorescent label, a colorimetric label, etc.) that permits detection and/or quantification of probe annealed to the amplified polynucleotide.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### Example

### Patient Samples

Frozen breast tumor and matched normal tissues were obtained from the University of Minnesota Tissue Procurement Facility (TPF) (n=52). Samples were chosen randomly with breast cancer and available matched normal tissue being the only selection criteria (Table 1). Mammary reduction samples were used as non-cancer controls (n=28). These studies were performed in accordance with IRB guidelines (IRB study number 1003E78700). The breast cancer cell line panel 30-4500K was obtained from the ATCC and cultured as recommended (n=45). V. Polunovsky provided hTERT-HMEC cells. RNA isolation, cDNA synthesis, and qPCR procedures were performed as reported (Refsland et al., 2010 Nucleic Acids Res 38:4274-4284; primer and probe sequences are listed in Table 2). P-values were calculated using 2-tailed ANOVA in SSP. Microscopy studies (Stenglein and Harris, 2006 J Biol Chem 281:16837-16841), TK fluctuation analyses (Stenglein et al., 2010 Nat Struct Mol Biol 17:222-229), and 3D-PCR experiments (Stenglein et al., 2010 Nat Struct Mol Biol 17:222-229; Suspène et al., 2011 Proc Natl Acad Sci USA 108, 4858-4863) were done as described.

Minor modifications to published protocols and new procedures are detailed below.

TABLE 1
Breast cancer information.

| | | | | | Her2/ | | |
|---|---|---|---|---|---|---|---|
| Patient ID | Age | Ethnicity | ER | PR | neu | Type | Grade |
| P-7142 | 40 | Caucasian | n.a. | n.a. | n.a. | IDC | 3 |
| P-2248 | 51 | African | + | − | − | IDC | 2 |
| | | American | | | | | |
| P-2100 | 75 | Caucasian | + | + | n.a. | IDC | 2 |
| P-2250 | 76 | Caucasian | + | + | − | IDC | 2 |
| P-0480 | 51 | Caucasian | n.a. | n.a. | n.a. | IDC | 3 |
| P-2296 | 49 | Caucasian | + | + | − | IDC | 2 |
| P-9407 | 38 | Caucasian | + | + | − | IDC | 2 |
| P-2498 | n.a. | Caucasian | + | + | − | IDC | 2 |
| P-1827 | 37 | Caucasian | n.a. | n.a. | n.a. | IDC/ILC | 2 |
| P-2671 | 61 | Caucasian | + | + | − | ILC | 2 |
| P-7020 | 40 | Caucasian | n.a. | n.a. | n.a. | IDC/ILC | 1 |
| P-2388 | 47 | Caucasian | + | + | − | IDC | 1 |
| P-1552 | 58 | Caucasian | + | + | − | IDC | 3 |
| P-1792 | 44 | Caucasian | + | + | n.a. | DCIS | 1 |
| P-1969 | 77 | Caucasian | n.a. | n.a. | n.a. | IDC | 2 |
| P-0637 | 70 | Caucasian | + | − | + | IDC | 2 |
| P-1127 | 68 | Caucasian | n.a. | n.a. | n.a. | DCIS | 1 |
| P-1624 | 49 | Caucasian | n.a. | n.a. | n.a. | IDC | 2 |
| P-2659 | 58 | Caucasian | + | − | + | IDC | 3 |
| P-1674 | 64 | Caucasian | + | − | − | ILC | 2 |
| P-2083 | 39 | Caucasian | + | + | − | IDC | 2 |
| P-1656 | 74 | Caucasian | + | + | − | ILC | 2 |
| P-8887 | 45 | Native | + | + | − | LC | 2 |
| | | American | | | | | |
| P-1677 | 49 | Caucasian | + | + | − | IDC | 2 |
| P-1121 | 75 | Caucasian | + | + | − | ILC | 1 |
| P-2528 | 51 | Caucasian | + | + | n.a. | ILC | 2 |
| P-1360 | 66 | Caucasian | + | + | n.a. | IDC | 2 |
| P-1734 | 47 | Caucasian | + | + | − | IDC | 2 |
| P-1651 | 51 | Caucasian | + | + | n.a. | IMC | 2 |
| P-2009 | 62 | Caucasian | + | + | − | IDC | 1 |
| P-1460 | 62 | Caucasian | + | + | + | IDC | 3 |
| P-0121 | 77 | Caucasian | + | + | n.a. | IDC | 2 |
| P-1367 | 43 | Caucasian | + | + | − | IDC | 2 |
| P-8277 | 54 | Caucasian | + | − | n.a. | IDC | 1 |
| P-9378 | 68 | Caucasian | + | − | − | ILC | 2 |
| P-1684 | 45 | Caucasian | + | + | − | IDC/ILC | 2 |
| P-1094 | 51 | Caucasian | + | + | n.a. | IDC | 2 |
| P-1017 | 40 | Caucasian | n.a. | n.a. | + | IDC | 3 |
| P-6841 | 68 | Caucasian | + | + | + | IDC | |
| P-0385 | 56 | Caucasian | + | + | − | ILC | 2 |
| P-1441 | 70 | Caucasian | − | − | − | IDC | 3 |
| P-0504 | 56 | Caucasian | + | − | − | IDC | 2 |
| P-0656 | 39 | Caucasian | − | − | + | IDC | 3 |
| P-8364 | 42 | Caucasian | n.a. | n.a. | n.a. | DCIS | 1 |
| P-7671 | 48 | Caucasian | + | − | n.a. | DCIS | 1 |
| P-9170 | 55 | Caucasian | n.a. | n.a. | n.a. | IDC | 2 |
| P-2625 | 72 | Caucasian | + | + | + | IDC | 3 |
| P-1257 | 77 | Caucasian | + | − | + | IDC | 2 |
| P-1150^{#} | 30 | Caucasian | + | + | ? | IDC | 3 |
| P-9773 | 37 | Caucasian | − | − | − | IDC | 3 |
| P-9169 | 62 | Caucasian | n.a. | n.a. | n.a. | IDC/ILC | 1 |
| P-9863 | 46 | Caucasian | + | + | − | IDC | 2 |

*Listed in order from A3B^{null} to A3B^{high} as in FIG. 2.
^{#}Male patient;
DCIS—Ductal carcinoma in situ;
IDC—Invasive ductal carcinoma;
ILC—Invasive lobular carcinoma;
IDC/ILC—Invasive ductal carcinoma with lobular features;
IMC—Invasive mucinous carcinoma;
n.a.—Not available.

TABLE 2
Quantitative PCR primer and probe information.

| | | 5′ | | SEQ | 3′ | | SEQ | | | SEQ |
|---|---|---|---|---|---|---|---|---|---|---|
| Gene | mRNA NCBI | Primer | | ID | Primer | | ID | Probe | | ID |
| Symbol | Accession | Name | Seq (5′-3′) | NO: | Name | Seq (5′-3′) | NO: | Name | Seq^{a} | NO: |
| APOBEC3s | | | | | | | | | | |
| APOBEC3A | NM_145699 | RSH2742 | gagaagggacaagcacatgg | 11 | RSH2743 | tggatccatcaagtgtctgg | 23 | UPL26 | ctgggctg | 35 |
| APOBEC3B | NM_004900 | RSH3220 | gaccctttggtccttcgac | 12 | RSH3221 | gcacagccccaggagaag | 24 | UPL1 | cctggagc | 36 |
| APOBEC3C | NM_014508 | RSH3085 | agcgcttcagaaaagagtgg | 13 | RSH3086 | aagtttcgttccgatcgttg | 25 | UPL155 | ttgccttc | 37 |
| APOBEC3D | NM_152426 | RSH2749 | acccaaacgtcagtcgaatc | 14 | RSH2750 | cacatttctgcgtggttctc | 26 | UPL51 | ggcaggag | 38 |
| APOBEC3F | NM_145298 | RSH2751 | ccgtttggacgcaaagat | 15 | RSH2752 | ccaggtgatctggaaacactt | 27 | UPL27 | gctgcctg | 39 |
| APOBEC3G | NM_021822 | RSH2753 | ccgaggacccgaaggttac | 16 | RSH2754 | tccaacagtgctgaaattcg | 28 | UPL79 | ccaggagg | 40 |
| APOBEC3H | NM_181773 | RSH2757 | agctgtggccagaagcac | 17 | RSH2758 | cggaatgtttcggctgtt | 29 | UPL21 | tggctctg | 41 |
| AID | NM_020661 | RSH3066 | gactttggttatcttcgcaat | 18 | RSH3067 | aggtcccagtccgagatgta | 30 | UPL69 | ggaggaag | 42 |
| | | | aaga | | | | | | | |
| APOBEC1 | NM_001644 | RSH3068 | gggaccttgttaacagtgga | 19 | RSH3069 | ccaggtgggtagttgacaaaa | 31 | UPL67 | tgctggag | 43 |
| | | | gt | | | | | | | |
| APOBEC2 | NM_006789 | RSH3070 | aagtagggcaactgggcttt | 20 | RSH3071 | ggctgtacatgtcattgctg | 32 | UPL74 | ctgctgcc | 44 |
| | | | | | | tc | | | | |
| APOBEC4 | NM_203454 | RSH3072 | ttctaacacctggaatgtga | 21 | RSH3073 | tttactgtcttctagctgca | 33 | UPL80 | cctggaga | 45 |
| | | | tcc | | | aacc | | | | |
| Reference | | | | | | | | | | |
| Gene | | | | | | | | | | |
| TBP | NM_003194 | RSH3231 | cccatgactcccatgacc | 22 | RSH3232 | tttacaaccaagattcactg | 34 | UPL51 | ggcaggag | 46 |
| | | | | | | tgg | | | | |

^{a}It is not known whether probes from the Universal Probe Library (UPL) correspond to the coding or template DNA strands of their target sequences (Roche; Madison, WI proprietary information).

### Cell Lines

Cell lines for pilot studies (MDA-MB-468, MCF-7, ZR-75-1, MDA-MB-231, T47D, BT-549, and MCF-10A) were kind gifts from Drs. Carol Lange and Douglas Yee (University of Minnesota). These lines were only used for the initial pilot study (FIG. 1). Subsequent experiments were performed using the ATCC lines. A panel of 45 different breast cell lines was obtained from ATCC (catalog number 30-4500K). Human mammary epithelial cells immortalized by human telomerase transduction (hTERT-HMECs) were a kind gift from Dr. Vitaly Polunovsky (University of Minnesota). All cell lines were cultured per ATCC recommendations.

### DNA Constructs

A3B shRNA and shControl lentiviral constructs were obtained from Open Biosystems. Helper plasmids pdelta-NRF, containing HIV-1-derived gag, pol, rev, and tat genes, and pMDG, containing the VGV-G env gene, were provided by Dr. Nik Somia (University of Minnesota). Stocks were prepared by co-transfecting HEK293T cells, harvesting cell-free supernatants, and concentrating by centrifugation (14,000×g for two hours). Stable breast cancer cell transductants were selected with puromycin (1 μg/mL).

The universal uracil DNA glycosylase inhibitor protein, Ugi, was expressed using a pcDNA3.1 (Clontech)-based expression plasmid as described (Klemm et al., 2009 Cancer Cell 16:232-245).

RNA Isolation, cDNA Synthesis, and qPCR

Total RNA was isolated from flash-frozen mammary tissue using RNeasy Lipid Tissue Mini Kits (Qiagen; Valencia, Calif.). 100 mg flash-frozen tissue was disrupted by a 3 hour water bath sonication in 1 mL of Qiazol Lysis Reagent (Qiagen; Valencia, Calif.). Total RNA was isolated using phenol/chloroform extraction and column purification as described by the manufacturer. Total RNA was isolated from cell lines by first disrupting 5 million cells using Qiashredder columns followed immediately by column purification using RNeasy Mini Kits (Qiagen; Valencia, Calif.). Quantitative RT-PCR was performed as described (Refsland et al., 2010 Nucleic Acids Res 38:4274-4284). Briefly, 1 μg of total RNA was used to synthesize cDNA using avian myeloblastosis virus reverse transcriptase (AMV RT; Roche; Madison, Wis.; Madison, Wis.) and random hexameric primers. cDNAs levels were quantified by PCR using a Roche Lightcycler 480 (Roche; Madison, Wis.) instrument according to manufacturer's protocols. cDNA was synthesized and qPCR performed in triplicate for each sample, and the means and standard deviations for each triplicate are reported. Data were normalized to the expression of the constitutive housekeeping gene TATA-binding protein (TBP). Primer and probe sequences are listed in Table 2.

### Microscopy

Microscopy procedures (Maul et al., 2011 Nat Immunol 12:70-76) were adapted for breast cancer cell lines. Briefly, the breast cancer cell-lines HCC1569, MDA-MB-453 and MDA-MB468 were added to LabTek chambered cover glasses (Nunc, ThermoFisher Scientific; Waltham, Mass.) at approximately 40% confluency and grown in standard cell culture for 24 hours. The cells were then transfected with Transit and either A3B- or A3F-eGFP constructs (Maul et al., 2011 Nat Immunol 12:70-76; Stenglein et al., 2008 J Virol 82:9591-9599) and incubated 48 hours. Before imaging the cells were treated with 0.1% Hoescht dye in PBC to stain the nuclei. A Deltavision deconvolution microscope (Applied Precision; Issaquah, Wash.) at 40× magnification was used to collect the images and deconvolution was performed using SoftWoRx, the Deltavision software (Applied Precision; Issaquah, Wash.).

### Cell Fractionation and Deaminase Activity Assays

Fractionation of breast cancer cell lysates was performed by incubating 10⁷ cells in 0.5 mL of hypotonic buffer (10 mM KCl, 10 mM Hepes, pH 7.4, 1.5 mM MgCl₂) on ice for one hour and then disrupting the plasma membrane by passage though a syringe (28 gauge) six times (modified from Abmayr et al., 2006 “Preparation of nuclear and cytoplasmic extracts from mammalian cells” in Curr Protoc Mol Biol Chapter 12, Unit 12.11). The nuclei were collected by centrifugation at low speed (600×g), washed with hypotonic buffer, and lysed by sonication in lysis buffer (25 mM Hepes, pH7.4, 250 mM NaCl, 10% glycerol, 0.5% Triton X-100, 1 mM EDTA, 1 mM MgCl₂, 1 mM ZnCl₂). The cytoplasmic and nuclear lysates from approximately 100,000 cells were loaded onto a 4-15% acrylimide gel and separated before transfer to pvdf. Fractionation was assessed by probing with anti-histone3 (1:2000) and anti-A3F (1:1000, visualized by fluorescent Licor anti-mouse 800 and anti-rabbit 680 (both 1:5000). Cell lysates, cytoplasmic fractions, or nuclear fractions were subjected to a fluorescence-based single-stranded DNA cytosine deaminase activity assay as described (Thielen et al., 2007 PLoS Pathol. 3:1320-1334). Briefly, serial dilutions of lysates were incubated for two hours at 37° C. with a DNA oligonucleotide 5′-(6-FAM)-AAATTCTAATAGATAATGTGA-(TAMRA)-3′ (SEQ ID NO:47). FRET occurs between the fluorophores, decreasing FAM fluorescence. If cytidine deaminase activity is present in the lysates, the single cytidine is converted to uridine, which is then excised by uracil DNA glycosylase (New England Biolabs Inc.; Ipswish, Mass.). Resulting abasic sites were subjected to hydrolytic cleavage by adding 0.1 M NaOH. Once cleaved, the FAM and TAMRA labels are physically separated, FRET diminishes, and FAM fluorescence increases. Fluorescence is measured on the SynergyMx instrument (BioTek Instruments, Inc.; Winooksi, Vt.) with excitation at 490 nm and detection at 517 nm.

### Genomic Uracil Quantification Experiments

After shA3B or shControl transduction and 21 days continuous culture, genomic DNA was prepared, spiked with heavy (+6)-labeled uracil (C¹³ and N¹⁵ labeled; Cambridge Isotopes; Andover, Mass.) and treated with uracil DNA glycosylase (New England Biolabs Inc.; Ipswish, Mass.). Heavy-labeled and excised uracils were recovered using 3,000 MWCO spin columns (Pall Scientific; Port Washington, N.Y.). Uracils were further purified and concentrated by solid phase extraction using Carbograph SPE columns (Grace Davidson Discovery Science; Deerfield, Ill.). Each sample was eluted in LS-MS methanol, dried down under vacuum, and resuspended in 32 μL of LC-MS water with 0.1% formic acid. 8 μL of each sample was injected into a Thermo Scientific TSQ Quantum Ultra AM triple quadrupole mass spectrometer coupled to a Waters nanoACQUITY HPLC. Samples were eluted from the Hypercarb (ThermoFisher Scientific; Waltham, Mass.) column using a gradient of acetonitrile and water containing 0.1% formic acid. Excised uracils were quantified by summing the signal from molecular ions with M/Z ratios of 113.1, 70.05, and 96.05. The internal standard was quantified by summing the signal for ions with M/Z ratios of 119.1, 74.05, and 101.05. Final values for uracil quantification were calculated using each heavy internal standard as reference.

### DNA Sequencing

DNA was harvested from Ugi expressing MDA-MB-453 and HCC1569 cell lines and diluted to 10 ng/uL. Outer PCR with Phusion polymerase was performed using primers fwd 5′-TTTATCCATCCCATCACACCCTCAG-3′ (SEQ ID NO:1) and rev 5′-CCACTGACAACCACCCTTAACC-3′ (SEQ ID NO:2) for TP53 and fwd 5′-CCGTATAGCGTAAATTCCCAGA-3′ (SEQ ID NO:3) and rev 5′-CTGTTTTCCAGGGACTGAGG-3′ (SEQ ID NO:4) for PTEN ten cycles of 98° C. for 30 seconds, 50° C. for 30 seconds and 72° C. for 30 seconds. Inner PCR with Phusion polymerase was performed using primers fwd 5′-CAACTCTGTCTCCTTCCTCTTCC-3′ (SEQ ID NO:5) and rev 5′-AGACCTCAGGCGGCTCATAG-3′ (SEQ ID NO:6) for TP53 and fwd 5′-TTCTGAGGTTATCTTTTTACCACA-3′ (SEQ ID NO:7) and rev 5′-GGAAGAGGAAAGGAAAAACATC-3′ (SEQ ID NO:8) for PTEN thirty-five cycles of 98° C. for 30 seconds, 55° C. for 30 seconds and 72° C. for 45 seconds. The PCR product was purified using the PCR purification kit (Epoch Life Science; Missouri City, Tex.) and used for blunt end cloning into the vector, pJET followed by sequencing from the T7 promoter at the University of Minnesota Biomedical Genomics Center. Sequences were aligned and analyzed using Sequencher software.

Alternatively, DNA was harvested from HCC1569 cells transduced with shA3B or shControl lentiviruses and diluted to 10 ng/μl. 3D-PCR experiments (Stenglein et al., 2010 Nat Struct Mol Biol 17:222-229; Suspène et al., 2011 Proc Natl Acad Sci USA 108:4858-4863) were done as described using Taq polymerase (Denville Scientific; South Plainfield N.J.). TP53 outer PCR was done using primers 5′-GAGCTGGACCTTAGGCTCCAGAAAGGACAA-3′ (SEQ ID NO:48) and 5′-GCTGGTGTTGTTGGGCAGTGCTAGGAA-3′ (SEQ ID NO:49) and inner PCR with 5′-TTCTCTTTTCCTATCCTGAGTAGTGGTAA-3′ (SEQ ID NO:50) and 5′-AAAGGTGATAAAAGTGAATCTGAGGCATAA-3′ (SEQ ID NO:51). c-MYC outer PCR was done using primers 5′-ATGCCCCTCAACGTTAGCTTCACCAA-3′ (SEQ ID NO:52) and 5′-CTGGATGATGATGTTTTTGATGAA-3′ (SEQ ID NO:53) and inner PCR primers 5′-ACGAGGAGGAGAACTTCTACCAGCA-3′ (SEQ ID NO:54) and 5′-TCTCGTCGTCCGGGTCGCAGATGAA-3′ (SEQ ID NO:55). CDKN2B outer PCR was done using primers 5′-TGTGTGCCAGGTTGCCACTCTCA-3′ (SEQ ID NO:56) and 5′-AAGCCCAAGTACTGCCTGGGGA-3′ (SEQ ID NO:57) and inner PCR primers 5′-CCCAGTAGGCGGAGCAGAGGA-3′ (SEQ ID NO:58) and 5′-AGGGAGCCGCCAGGAATGCT-3′ (SEQ ID NO:59). The denaturation gradient for each gene is shown in FIG. 12d. PCR products were analyzed by agarose gel electrophoresis, visualized with ethidium bromide, purified using a PCR purification kit (Epoch Life Science; Missouri City, Tex.), blunt-end cloned into NET (Feimentas; a part of ThermoFosher Scientific; Waltham, Mass.), and sequenced using the T7 promoter primer (University of Minnesota Biomedical Genomics Center). Sequences were aligned and analyzed with Sequencher software (Gene Codes Corporation; Ann Arbor, Mich.).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

All headings are for the convenience of the reader and should not be used to limit the meaning of the text that follows the heading, unless so specified.

Sequence Listing Free Text

SEQ ID NO: 1
TTTATCCATCCCATCACACCCTCAG
SEQ ID NO: 2
CCACTGACAACCACCCTTAACC
SEQ ID NO: 3
CCGTATAGCGTAAATTCCCAGA
SEQ ID NO: 4
CTGTTTTCCAGGGACTGAGG
SEQ ID NO: 5
CAACTCTGTCTCCTTCCTCTTCC
SEQ ID NO: 6
AGACCTCAGGCGGCTCATAG
SEQ ID NO: 7
TTCTGAGGTTATCTTTTTACCACA
SEQ ID NO: 8
GGAAGAGGAAAGGAAAAACATC
SEQ ID NO: 9
MNPQIRNPME RMYRDTFYDN FENEPILYGR SYTWLCYEVK IKRGRSNLLW
DTGVFRGQVY FKPQYHAEMC FLSWFCGNQL PAYKCFQITW FVSWTPCPDC
VAKLAEFLSE HPNVTLTISA ARLYYYWERD YRRALCRLSQ AGARVTIMDY
EEFAYCWENF VYNEGQQFMP WYKFDENYAF LHRTLKEILR YLMDPDTFTF
NFNNDPLVLR RRQTYLCYEV ERLDNGTWVL MDQHMGFLCN EAKNLLCGFY
GRHAELRFLD LVPSLQLDPA QIYRVTWFIS WSPCFSWGCA GEVRAFLQEN
THVRLRIFAA RIYDYDPLYK EALQMLRDAG AQVSIMTYDE FEYCWDTFVY
RQGCPFQPWD GLEEHSQALS GRLRAILQNQ GN
SEQ ID NO: 10
acagagcttc aaaaaaagag cgggacaggg acaagcgtat ctaagaggct gaacatgaat
ccacagatca gaaatccgat ggagcggatg tatcgagaca cattctacga caactttgaa
aacgaaccca tcctctatgg tcggagctac acttggctgt gctatgaagt gaaaataaag
aggggccgct caaatctcct ttgggacaca ggggtctttc gaggccaggt gtatttcaag
cctcagtacc acgcagaaat gtgcttcctc tcttggttct gtggcaacca gctgcctgct
tacaagtgtt tccagatcac ctggtttgta tcctggaccc cctgcccgga ctgtgtggcg
aagctggccg aattcctgtc tgagcacccc aatgtcaccc tgaccatctc tgccgcccgc
ctctactact actgggaaag agattaccga agggcgctct gcaggctgag tcaggcagga
gcccgcgtga cgatcatgga ctatgaagaa tttgcatact gctgggaaaa ctttgtgtac
aatgaaggtc agcaattcat gccttggtac aaattcgatg aaaattatgc attcctgcac
cgcacgctaa aggagattct cagatacctg atggatccag acacattcac tttcaacttt
aataatgacc ctttggtcct tcgacggcgc cagacctact tgtgctatga ggtggagcgc
ctggacaatg gcacctgggt cctgatggac cagcacatgg gctttctatg caacgaggct
aagaatcttc tctgtggctt ttacggccgc catgcggagc tgcgcttctt ggacctggtt
ccttctttgc agttggaccc ggcccagatc tacagggtca cttggttcat ctcctggagc
ccctgcttct cctggggctg tgccggggaa gtgcgtgcgt tccttcagga gaacacacac
gtgagactgc gcatcttcgc tgcccgcatc tatgattacg accccctata taaggaggcg
ctgcaaatgc tgcgggatgc tggggcccaa gtctccatca tgacctacga tgagtttgag
tactgctggg acacctttgt gtaccgccag ggatgtccct tccagccctg ggatggacta
gaggagcaca gccaagccct gagtgggagg ctgcgggcca ttctccagaa tcagggaaac
tgaaggatgg gcctcagtct ctaaggaagg cagagacctg ggttgagcag cagaataaaa
gatcttcttc caagaaatgc aaacagaccg ttcaccacca tctccagctg ctcacagaca
ccagcaaagc aatgtgctcc tgatcaagta gattttttaa aaatcagagt caattaattt
taattgaaaa tttctcttat gttccaagtg tacaagagta agattatgct caatattccc
agaatagttt tcaatgtatt aatgaagtga ttaattggct ccatatttag actaataaaa
cattaagaat cttccataat tgtttccaca aacact
SEQ ID NO: 11
gagaagggac aagcacatgg
SEQ ID NO: 12
gaccctttgg tccttcgac
SEQ ID NO: 13
agcgcttcag aaaagagtgg
SEQ ID NO: 14
acccaaacgt cagtcgaatc
SEQ ID NO: 15
ccgtttggac gcaaagat
SEQ ID NO: 16
ccgaggaccc gaaggttac
SEQ ID NO: 17
agctgtggcc agaagcac
SEQ ID NO: 18
gactttggtt atcttcgcaa taaga
SEQ ID NO: 19
gggaccttgt taacagtgga gt
SEQ ID NO: 20
aagtagggca actgggcttt
SEQ ID NO: 21
ttctaacacc tggaatgtga tcc
SEQ ID NO: 22
cccatgactc ccatgacc
SEQ ID NO: 23
tggatccatc aagtgtctgg
SEQ ID NO: 24
gcacagcccc aggagaag
SEQ ID NO: 25
aagtttcgtt ccgatcgttg
SEQ ID NO: 26
cacatttctg cgtggttctc
SEQ ID NO: 27
ccaggtgatc tggaaacact t
SEQ ID NO: 28
tccaacagtg ctgaaattcg
SEQ ID NO: 29
cggaatgttt cggctgtt
SEQ ID NO: 30
aggtcccagt ccgagatgta
SEQ ID NO: 31
ccaggtgggt agttgacaaa a
SEQ ID NO: 32
ggctgtacat gtcattgctg tc
SEQ ID NO: 33
tttactgtct tctagctgca aacc
SEQ ID NO: 34
tttacaacca agattcactg tgg
SEQ ID NO: 35
ctgggctg
SEQ ID NO: 36
cctggagc
SEQ ID NO: 37
ttgccttc
SEQ ID NO: 38
ggcaggag
SEQ ID NO: 39
gctgcctg
SEQ ID NO: 40
ccaggagg
SEQ ID NO: 41
tggctctg
SEQ ID NO: 42
ggaggaag
SEQ ID NO: 43
tgctggag
SEQ ID NO: 44
ctgctgcc
SEQ ID NO: 45
cctggaga
SEQ ID NO: 46
ggcaggag
SEQ ID NO: 47
5′-(6-FAM)-AAATTCTAATAGATAATGTGA-(TAMRA)-3′
SEQ ID NO: 48
GAGCTGGACCTTAGGCTCCAGAAAGGACAA
SEQ ID NO: 49
GCTGGTGTTGTTGGGCAGTGCTAGGAA
SEQ ID NO: 50
TTCTCTTTTCCTATCCTGAGTAGTGGTAA
SEQ ID NO: 51
AAAGGTGATAAAAGTGAATCTGAGGCATAA
SEQ ID NO: 52
ATGCCCCTCAACGTTAGCTTCACCAA
SEQ ID NO: 53
CTGGATGATGATGTTTTTGATGAA
SEQ ID NO: 54
ACGAGGAGGAGAACTTCTACCAGCA
SEQ ID NO: 55
TCTCGTCGTCCGGGTCGCAGATGAA
SEQ ID NO: 56
TGTGTGCCAGGTTGCCACTCTCA
SEQ ID NO: 57
AAGCCCAAGTACTGCCTGGGGA
SEQ ID NO: 58
CCCAGTAGGCGGAGCAGAGGA
SEQ ID NO: 59
AGGGAGCCGCCAGGAATGCT
SEQ ID NO: 60
ATGTCATTACATAT
SEQ ID NO: 61
ATATGTAATGACAT
SEQ ID NO: 62
ATGTUATTAUATAT
SEQ ID NO: 63
ATATGTAATGAUAT
SEQ ID NO: 64
ATGTnATTAnATAT
SEQ ID NO: 65
ATATGTAATGAnAT
SEQ ID NO: 66
ATATTATTATATAT
SEQ ID NO: 67
ATATATAATAATAT
SEQ ID NO: 68
MNPQIRNPME RMYRDTFYDN FENEPILYGR SYTWLCYEVK IKRGRSNLLW
DTGVFRGQVY FKPQYHAEMC FLSWFCGNQL PAYKCFQITW FVSWTPCPDC
VAKLAEFLSE HPNVTLTISA ARLYYYWERD YRRALCRLSQ AGARVTIMDY
EEFAYCWENF VYNEGQQFMP WYKFDENYAF LHRTLKEILR YLMDPDTFTF
NFNNDPLVLR RRQTYLCYEV ERLDNGTWVL MDQHMGFLCN EAKNLLCGFY
GRHAELRFLD LVPSLQLDPA QIYRVTWFIS WSPCFSWGCA GEVRAFLQEN
THVRLRIFAA RIYDYDPLYK EALQMLRDAG AQVSIMTYDE FEYCWDTFVY
RQGCPFQPWD GLEEHSQALS GEAAGHSPES GKLKDGPQSL RKAETWVEQQ
NKRSSSKKCK QTVHHHLQLL TDTSKAMCS

What is claimed is:

1. A method comprising:
measuring expression of APOBEC3B in a biological sample obtained from a patient; and
identifying the patient as having or at risk for having cancer if the measured expression of APOBEC3B is greater than a predetermined reference level of expression.

2. The method of claim 1 wherein the predetermined reference level of expression comprises the level of APOBEC3B expression in a normal cell.

3. The method of claim 1 wherein the cancer comprises breast cancer.

4. The method of claim 1 wherein measuring expression of APOBEC3B comprises quantitative PCR.

5. The method of claim 4 wherein the quantitative PCR comprises annealing a polynucleotide from the biological sample with at least one primer that comprises the nucleotide sequence of SEQ ID NO:12 or the nucleotide sequence of SEQ ID NO:24.

6. The method of claim 5 wherein the quantitative PCR comprises:
annealing a polynucleotide from the biological sample with the nucleotide sequence of SEQ ID NO:12; and
annealing a polynucleotide from the biological sample with the nucleotide sequence of SEQ ID NO:24.

7. An isolated polynucleotide comprising the nucleotide sequence of SEQ ID NO:12.

8. An isolated polynucleotide comprising the nucleotide sequence of SEQ ID NO:24.

9. A kit comprising in separate containers:
a first primer that anneals to a first strand of a polynucleotide that encodes APOBEC3B; and
a second primer that anneals to a second strand of a polynucleotide that encodes APOBEC3B.

10. The kit of claim 9 wherein the first primer comprises the nucleotide sequence of SEQ ID NO:12 and the second primer comprises the nucleotide sequence of SEQ ID NO:24.

11. The kit of claim 9 further comprising in a separate container a probe that anneals to a nucleotide sequence amplified by polymerase chain reaction using the first primer and the second primer.

12. The kit of claim 11 wherein the probe comprises the nucleotide sequence of SEQ ID NO:36.

13. A method comprising:
measuring expression of APOBEC3B in a biological sample obtained from a patient; and
determining a therapeutic parameter if the measured expression of APOBEC3B is greater than a predetermined reference level of expression, wherein the therapeutic parameter comprises a probability of outcome, likelihood of recurrence, likelihood of therapy resistance, or any combination thereof.

14. The method of claim 13 wherein the predetermined reference level of expression comprises the level of APOBEC3B expression in a normal cell.

15. The method of claim 13 wherein measuring expression of APOBEC3B comprises quantitative PCR.

16. The method of claim 15 wherein the quantitative PCR comprises annealing a polynucleotide from the biological sample with at least one primer that comprises the nucleotide sequence of SEQ ID NO:12 or the nucleotide sequence of SEQ ID NO:24.

17. The method of claim 16 wherein the quantitative PCR comprises:
annealing a polynucleotide from the biological sample with the nucleotide sequence of SEQ ID NO:12; and
annealing a polynucleotide from the biological sample with the nucleotide sequence of SEQ ID NO:24.
